(19) United States
(12) Patent Application Publication
(10) Pub. No.: US 2006/0047108 A1
(43) Pub. Date: Mar. 2, 2006

(54) Synthesis of idarubicin aglycone

(76) Inventors: Marco Villa, Milano (IT); Roberto Arosio, Civate (LC) (IT); Roberta Fretta, Collegno (TO) (IT); Nicola Diulgheroff, Torino (IT)

Correspondence Address: KENYON & KENYON, ONE BROADWAY, NEW YORK, NY, 10004, US

(21) Appl. No.: 11/210,605

(22) Filed: Aug. 23, 2005

Related U.S. Application Data
(60) Provisional application No. 60/604,038, filed on Aug. 23, 2004.
(60) Provisional application No. 60/606,813, filed on Sep. 1, 2004.

(51) Int. Cl. C07H 15/24; C07C 46/00

(52) U.S. Cl. 536/6.4; 552/216

(57) ABSTRACT

The present invention provides a new method of producing high quality idarubicin aglycone from 4-protected demethoxydaunomycinones such as 4-demethoxydaunomycinone-4-triflate.

### CROSS REFERENCE TO RELATED APPLICATIONS

This application claims the benefit of U.S. provisional application Nos. 60/604,038, filed Aug. 23, 2004; and 60/606,813, filed Sep. 1, 2004; the contents of all of which are incorporated herein by reference.

### FIELD OF THE INVENTION

The present invention relates to an improved method for preparing idarubicin aglycone.

### BACKGROUND OF THE INVENTION

(7S, 9S) 9-acetyl-7,8,9,10-tetrahydro-6,7,9,11-tetrahydroxy-5,12-naphthacenedione, (Idarubicin aglycon or 4-demethoxydaunomycinone) having the formula, 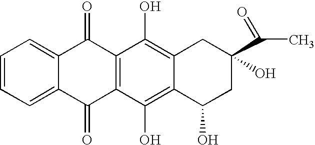
is a derivative of (7S, 9S)-7-[(3-amino-2,3,6-trideoxy-(alpha)-L-1yxo-hexopyranosyl)oxy]-7,8,9,10-tetrahydro-6,9,11-trihydroxy-9-acetyl-5,12-naphthacenedione having the following formula.

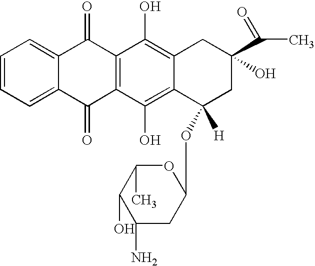

International Patent Publication WO 01/87814 discloses a process for preparing 4-demethyldaunomycinone-4-triflate followed by its reduction to idarubicin aglycone. WO 01/87814 describes reduction of 4-demethyldaunomycinone-4-triflate (0.012 moles) with triethylamine (0.045 moles), 99% formic acid (0.040 moles), 1,1′bis-(diphenylphosphino)ferrocene (0.0005 moles) and Pd(OAc)₂ (0.0005 moles), in dioxane, at 40° C. The yield reported is 66%, but no information about the product purity, or description of possible purification steps, are given. The products of the reaction described in WO 01/87814 would be expected to be very difficult to clean up, likely requiring chromatography steps that are not very convenient to carry out on an industrial scale.

U.S. Pat. No. 5,103,029 discloses a process in which 4-demethoxydaunomycinone is obtained with a yield of 71.6% and 98% purity (HPLC), but only after chromatographic purification of the final crude product. The necessity for significant purification steps was not unexpected, since impurities (e.g., 4-demethyldaunomycinone, and products of aromatization of the anthracycline A ring) typically form when the described reductions are conducted on this kind of substrate. Such impurities are usually present in large amounts and/or are not easily removed from the crude product because of their chemical and physical properties, usually requiring chromatographic purifications. Thus, according to these prior art processes, purification steps applied to the crude 4-demethoxydaunomycinone are usually necessary, and strongly reduce the overall yield of the processes.

U.S. Pat. No. 5,015,745 discloses a method of making 4-demethoxydaunomycinone from demethyldaunomycinone comprising the steps of protecting the 13-keto group, sulfonylating the 4-OH group, reacting the sulfonylated compound to produce an amine at the 4 position, diazotizing the 4-amine, and reducing under mild conditions to give the final demethoxy compound.

Therefore, there is a need for a new process for the reduction of the triflate group to obtain pure idarubicin aglycone, wherein the amount of the undesired byproducts is small.

### SUMMARY OF THE INVENTION

One aspect of the present invention is a process for the preparation of idarubicin aglycone (4-demethoxydaunomycinone) of formula I 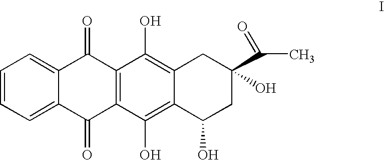
comprising the steps of
(a) combining the corresponding sulfonate of formula II 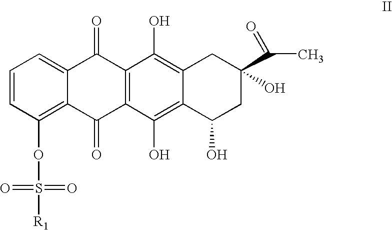
with a first polar aprotic organic solvent and with a metal catalyst or co-catalyst to obtain a first solution;
(b) combining about 0.9 to about 1.3 mole equivalents of the silyl reagent of the formula R₂R₃R₄SiH with a base and a second polar aprotic organic solvent, with or without a protic solvent to obtain a second solution;
(c) adding the second solution of step (b) to the first solution of step (a) to obtain a mixture, and
(d) adding a solution of the silyl reagent of the formula R₂R₃R₄SiH in a second polar aprotic organic solvent to the mixture of step (c), when no more than 96% area by HPLC of the sulfonate of formula II has reacted, and
(e) maintaining the mixture obtained in step (d) for at least 20 minutes;
(f) quenching the mixture maintained in step (e); and
(g) recovering the idarubicin aglycone of formula I;

wherein R₁ is C₁₋₁₀ alkyl, C₁₋₁₀ alkyl substituted with halogens, an aryl group, an aryl group substituted with halogen or an electron withdrawing group, and R₂, R₃, and R₄ are independently branched or linear C₁₋₄ alkyl, aryl, heteroaryl groups, or polymethylsiloxane.

Preferably, R₁ is C₁₋₁₀ alkyl fully substituted with halogens, and, more preferably, is CF₃.

Preferably, R₂, R₃ and R₄ are the same alkyl groups, and, more preferably, R₂, R₃ and R₄ are ethyl groups.

Preferably, a co-catalyst is used in step (a).

Preferably, the silyl reagent of the formula R₂R₃R₄SiH is used in step (b) in an amount of about 1.15 mole equivalent per mole equivalent of the sulfonate of the formula II.

Preferably, the second solution of step (b) is added to the first solution of step (a) in a dropwise manner. More preferably, the dropwise addition is done over a period of about 20 minutes to about 1.5 hours.

Preferably, the solution of step (b) also contains a protic solvent. Preferably, the protic solvent is either a C₁₋₅ alcohol or water. Preferably, the C₁₋₅ alcohol is methanol. The more preferred protic solvent is water.

Preferably, the amount of the protic solvent is of about 0.1 mole equivalents to about 5 mole equivalents per mole equivalent of the sulfonate of formula II.

Idarubicin aglycone of formula I may be purified by a process of crystallization from a mixture of a solvent and an anti-solvent. The solvent is preferably, a polar organic solvent, selected from the group consisting of dichloromethane, acetone, acetonitrile and THF. The anti-solvent is preferably a non-polar organic solvent, more preferably, diisopropylether or toluene. Most preferably, the mixture of a solvent and an anti solvent contains acetonitrile with diisopropylether or THF with toluene.

Idarubicin aglycone obtained by the above crystallization process contains less than about 1% area by HPLC, of undesired byproducts.

Preferably, idarubicin aglycone obtained by the above crystallization process contains less than about 0.1% area by HPLC of 4-hydroxy derivative of formula IV.

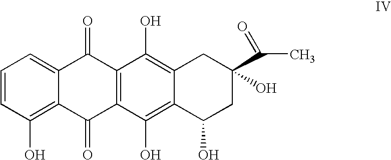

Another aspect of the present invention is idarubicin aglycone of formula I containing less than about 0.1% area by HPLC of 4-hydroxy derivative of formula IV.

Idarubicin aglycone obtained by the process of this invention may be converted to pharmaceutically acceptable salts of idarubicin, preferably, idarubicin hydrochloride.

### BRIEF DESCRIPTION OF THE DRAWINGS

FIG. 1: Structure and carbon skeleton numbering of idarubicin aglycone (7S, 9S) 9-acetyl-7,8,9,10-tetrahydro-6,7,9,11-tetrahydroxy-5,12-naphthacenedione.

FIG. 2: Structure and carbon skeleton numbering of 4-demethyldaunomycinone-4-triflate.

FIG. 3: Schematic flow diagram of a reaction of the present invention.

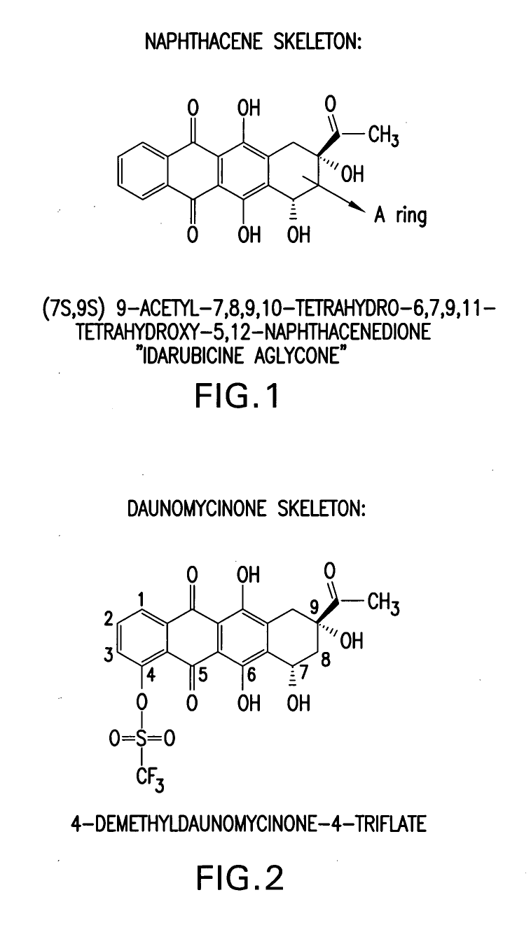

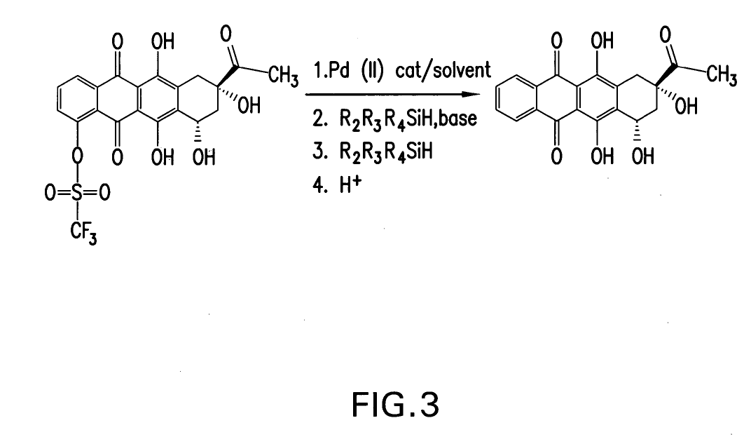

### DETAILED DESCRIPTION OF THE INVENTION

The term “heteroaryl” refers to an aryl group that includes from one to four heteroatoms, for example, pyrrole, furan, thiophene, imidazole, oxazole, thiazole, triazole, pyrazole, pyridine, pyrazine, pyridazine, pyrimidine, and the like. The term “heteroatom,” as used herein, means an atom of any element other than carbon or hydrogen.

As used herein, the term electron “withdrawing group” refers to a single atom or to a group of atoms that cause the electron density of the chemical bond to shift toward them, for example, NO₂, COOR, COOH, CO, SO₂, CO, and C₁₋₁₀ alkyl fully substituted with halogens.

As used herein, the term “weak organic base” refers to a negatively charged single atom or a group of atoms that have low affinity to H⁺, for example, pyridine, substituted pyridine, Et₃N, Bu₃N and ethyldiisopropylamine.

As used herein, the term “hindered organic base” refers to a negatively charged single atom or a group of atoms substituted with bulky groups, for example, Et₃N, 2,6-dimethylpyridine, diisopropylethylamine and tributylamine.

As used herein, the term “co-catalyst” refers to the non-active form of the catalyst, which is transformed to the active form by a reaction.

In the process of the present invention the 13-keto group of the 4-demethyldaunomycinone-4-triflate does not have to be protected prior to conversion of the 4-OH group to the 4-demethoxy, as in the prior art processes described in U.S. Pat. No. 5,015,745 and in European Patent Application 0 337 665.

In the process of the present invention, idarubicin aglycone (4-demethoxydaunomycinone) is obtained in much higher yields and with greater purity than with prior art methods. In particular, the formation of those impurities that typically occurs when reductive processes are conduced on this kind of substrate, represented by the compound of the formula III, 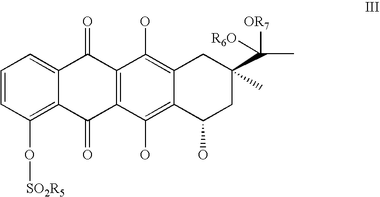
wherein, R₆ and R₇ can be the same, preferably C₁₋₁₀ alkyl or —CH₂—CH₂—, are drastically reduced. Under the conditions of the invention, impurities are present in minimal amounts, so that the crude final product does not need any subsequent onerous purification steps, such as chromatography. Thus, the high quality of the crude idarubicin aglycone allows the use of a very simple crystallization procedure—if needed—which leads to a high quality product in a very high yield.

The present invention provides a process for the preparation of pure idarubicin aglycone (4-demethoxydaunomycinone) of formula I 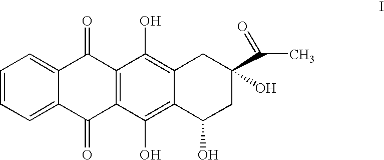
comprising the steps of
(a) combining the corresponding sulfonate of formula II, 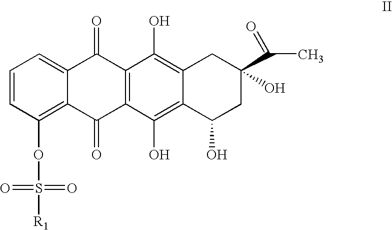
with a first polar aprotic organic solvent and with a metal catalyst or co-catalyst to obtain a first solution;
(b) combining about 0.9 to about 1.3 mole equivalents of the silyl reagent of the formula R₂R₃R₄SiH with a base, and a second polar aprotic organic solvent, with or without a protic solvent to obtain a second solution;
(c) adding the second solution of step (b) to the first solution of step (a) to obtain a mixture, and
(d) adding a solution of the silyl reagent of the formula R₂R₃R₄SiH in a second polar aprotic organic solvent to the mixture of step (c), when no more than 96% area by HPLC of the sulfonate of formula II has reacted; and
(e) maintaining the mixture obtained in step (d) for at least 20 minutes;
(f) quenching the mixture maintained in step (e), and
(g) recovering the idarubicin aglycone of the formula I; wherein R₁ is C₁₋₁₀ alkyl, C₁₋₁₀ alkyl substituted with halogen, an aryl group, an aryl group substituted with halogen or an electron withdrawing group; and R₂, R₃, and R₄ are independently branched or linear C₁₋₄ alkyl, aryl, heteroaryl groups or polymethylsiloxane.

Preferably, R₁ is C₁₋₁₀ alkyl fully substituted with halogen, and, more preferably, is CF₃. The compound of formula II corresponds to 4-demethyldaunomycinone-4-triflate having the following formula.

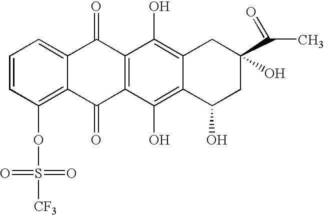

### 4-demethyldaunomycinone-4-triflate

Preferably, R₂, R₃ and R₄ are the same alkyl groups, and, more preferably, R₂, R₃ and R₄ are ethyl groups. Preferably, the compound of the formula R₂R₃R₄SiH is triethylsilane.

Preferably, Et₃SiH is commercially available.

The 4-demethoyldaunomycinone-4-triflate of formula II may be prepared, for example, according to the process disclosed in WO 01/87814.

The temperature of step (a) is preferably from about 10° C. to about 46° C., and, more preferably, is from about 15° C. to about 25° C.

The first polar aprotic organic solvent used in step (a) is selected from the group consisting of amide, ether and ketone. A preferred amide is dimethylformamide (DMF), dimethylacetamide, or N-methylpyrrolidinone. Preferably, the ether is a cyclic ether, more preferably, tetrahydrofuran (THF) or 2-methyl-THF. A preferred ketone is acetone. More preferably, the first polar aprotic organic solvent is DMF. Preferably, a co-catalyst is used in step (a).

The metal co-catalyst is preferably, dichlorobis(triphenylphosphine)Ni(II), dichlorobis(triphenylphosphine)Pd(II), or Pd(II)(OAc)₂ in the presence of triphenylphosphine, and, more preferably, is dichlorobis(triphenylphosphine)Pd(II), which is reduced in situ to a give the active catalyst [bis(triphenylphosphine)]Pd(0).

Preferably, the silyl reagent of formula R₂R₃R₄SiH is used in step (b) in an amount of about 1.15 mole equivalent per mole equivalent of the sulfonate of formula II.

The base used in step (b) is preferably, a weak organic base, more preferably, a hindered base, selected from the group consisting of pyridine, 2,6-dimethylpyridine, diisopropylethylamine, triethylamine, tributylamine, and imidazole. Most preferably, the base is 2,6-dimethylpyridine. The use of a weak base, such as 2,6-dimethylpyridine, instead of strong bases limits the amount of byproducts that can be produced in the reaction.

The second polar aprotic organic solvent in step (b) and in step (d) is preferably the same as the aprotic organic solvent in step (a); more preferably, the second polar aprotic organic solvent is DMF.

Preferably, the second solution of step (b) is added to the first solution of step (a) in a dropwise manner. More preferably, the dropwise addition was done over a period of about 20 minutes to about 1.5 hours.

The dropwise addition of the above solution allows an accurate control of the silane quantity present in the reaction mixture. An excess of silane at prolonged reaction times induces the formation of over-reduced species, while a lack of silane prevents the complete conversion of starting material. Therefore, the reaction progress is monitored by HPLC, and, when no more than 96% area by HPLC, of the sulfonate of formula II has reacted, a second amount of the silane solution is added.

Preferably, the solution of step (b) also contains a protic solvent to avoid the formation of some impurities, such as the 7-deoxy derivative of formula V. Preferably, the protic solvent is either C₁₋₅ alcohol or water. Preferably, the C₁₋₅ alcohol is methanol. The more preferred protic solvent is water.

Preferably, the amount of the protic solvent is about 0.1 mole equivalents to about 5 mole equivalents per mole equivalent of the sulfonate of formula II.

The solution of the silane of formula R₂R₃R₄SiH in the second polar aprotic organic solvent is added in step (d) to bring the reaction to completion.

The mixture obtained in step (d) is maintained preferably, for about 20 minutes to about 2 hours, depending on the reaction temperature.

Preferably, quenching may be done using an acidic aqueous solution, more preferably, HCl, acetic acid, or ammonium chloride. Most preferably, quenching may be done using aqueous HCl.

Idarubicin aglycone of formula I is recovered by any method known in the art, such as precipitating by the addition of water, filtering and washing the obtained solid with a polar organic solvent or with water.

Idarubicin aglycone of formula I may be further purified by a process of crystallization from a mixture of a solvent and an anti-solvent. The solvent is preferably, a polar organic solvent, selected from the group consisting of dichloromethane, acetone, acetonitrile and THF. The anti-solvent is preferably a non-polar organic solvent; more preferably, diisopropylether or toluene. Most preferably, the mixture of a solvent and an anti-solvent contains acetonitrile with diisopropylether or THF with toluene.

Idarubicin aglycone obtained by the above crystallization process contains less than about 1% area by HPLC, of undesired byproducts. Such byproducts may be one of: 4-hydroxy derivative of formula IV, 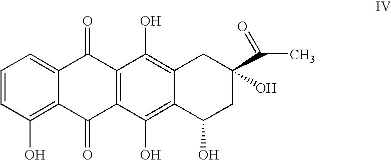
7-deoxy derivative of formula V 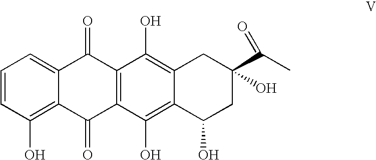
and idarubicin aglycone bis-anhydro of formula VI.

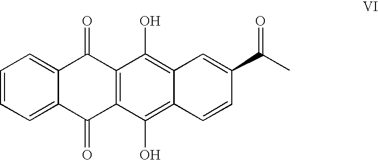

Preferably, idarubicin aglycone prepared by the above process contains less than about 0.1% of 4-hydroxy derivative of formula IV.

The present invention also provides idarubicin aglycone of formula I containing less than about 0.1% area by HPLC of 4-hydroxy derivative of formula IV.

Idarubicin aglycone obtained by the process of this invention may be converted to pharmaceutically acceptable salts of idarubicin preferably, idarubicin hydrochloride, for example, according to the process described in U.S. Pat. No. 4,077,988.

The processes of producing idarubicin aglycone described herein may be altered in size according to the amount of product desired or the nature of the equipment used. The amounts of reagents indicated in the Examples are based on a typical production batch, but such amounts can be varied, depending on the amount of product desired or the nature of the equipment used. Reaction temperatures, times, and quantities of chemicals indicated may be varied somewhat to increase process efficiency without adversely affecting product characteristics.

The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### EXAMPLES

### Example 1

A solution of 4-demethyldaunomycinone-4-triflate¹ (50.00 g, 96.7 mmol) and dichlorobis(triphenylphosphine)palladium(II) (1.36 g, 1.93 mmol) in dimethylformamide (1130 ml) was prepared at 24° C., under a nitrogen stream. A solution of triethylsilane (11.89 g, 102.2 mmol), 2,6-dimethylpyridine (11.93 g, 111.0 mmol) and water (4.35 g, 242.0 mmol) in dimethylformamide (1066 ml) was added dropwise in 1 h. The reaction was monitored by HPLC. When the conversion of 4-demethyldaunomycinone-4-triflate arrested (in about 8 h., with 90-98% of conversion), a solution of triethylsilane (0.24 g, 2.1 mmol) in dimethylformamide (14 ml) was added. After 2 h., the reaction mixture was treated with 37% hydrochloric acid (9.3 ml) and H₂O (4400 ml) was then added dropwise over 1.5 h. The crude product was recovered by filtration, washed with a mixture of H₂O (440 ml) and methanol (440 ml), and dried at 50° C. under vacuum. Crude 4-demethoxydaunomycinone (33.8 g, recovery 94.9%, HPLC purity 95.5%, containing 7-deoxy-4-demethoxydaunomycinone 0.4%, Idarubicin aglycone bis-anhydro 0.14% and 4-demethyl-daunomicinone 0.28%, which is the 4-hydroxy derivative, was suspended in methylene chloride (1010 ml) and acetone (440 ml) and the mixture was refluxed for 3 hours. Diisopropylether (930 ml) was then added dropwise, keeping the mixture under reflux. The mixture was concentrated under vacuum to a residue volume of 2.01, diisopropylether (1930 ml) was added dropwise and the resulting mixture was kept under reflux for 0.5 h. The mixture was then cooled to room temperature, kept at this temperature for 3 hours, and filtered to obtain 30.10 g of crystal 4-demethoxydaunomycinone. Overall yield 84.5%, HPLC purity 99% (containing 7-deoxy-4-demethoxydaunomycinone 0.14%, and 4-demethyl-daunomicinone 0.23%, which is the 4-hydroxy derivative).
¹Starting material contains 0.17% of 4-demethyl-daunomicinone, which is the 4-hydroxy derivative.

### Example 2

A solution of 4-demethyldaunomycinone-4-triflate¹ (50.00 g, 96.7 mmol) and dichlorobis(triphenylphosphine)palladium(II) (1.36 g, 1.93 mmol) in dimethylformamide (1130 ml) was prepared at 21° C., under a nitrogen stream. A solution of triethylsilane (11.24 g, 96.7 mmol), 2,6-dimethylpyridine (11.93 g, 111.0 mmol) and water (1.74 g, 96.7 mmol) in dimethylformamide (1066 ml) was added dropwise over 20 minutes. When the conversion of 4-demethyldaunomycinone-4-triflate arrested (95% conversion), a solution of triethylsilane (0.56 g, 4.84 mmol) in dimethylformamide (14 ml) was added. After 2 h., the work up of the mixture and purification of crude product (the crude containing: 7-deoxy-4-demethoxydaunomycinone 0.28% and4-demethyl-daunomicinone 0.31%) were accomplished as in Example 1, obtaining 31.08 g of crystal 4-demethoxydaunomycinone. Overall yield 88%, HPLC purity 98.5% (containing 7-deoxy-4-demethoxydaunomycinone 0.12% and 4-demethyl-daunomicinone 0.3%)
¹Starting material contains 0.25% of 4-demethyl-daunomicinone.

### Example 3

A solution of 4-demethyldaunomycinone-4-triflate¹ (50.00 g, 96.7 mmol) and dichlorobis(triphenylphosphine)palladium(II) (1.36 g, 1.93 mmol) in dimethylformamide (1130 ml) was prepared at 24° C., under a nitrogen stream. A solution of triethylsilane (10.68 g, 91.85 mmol), 2,6-dimethylpyridine (11.93 g, 111.0 mmol) and water (0.87 g, 48.4 mmol) in dimethylformamide (1066 ml) was added (all at once). When the conversion of 4-demethyldaunomycinone-4-triflate arrested (95% conversion), a solution of triethylsilane (0.56 g, 4.82 mmol) in dimethylformamide (14 ml) was added. After 2 h., the work up of the mixture and purification of crude product (purity 90.9% by HPLC, containing 7-deoxy-4-demethoxydaunomycinone 0.69%, Idarubicin aglycone bis-anhydro 0.2%, 4-demethyldaunomycinone-4-triflate 6.6% and 4-demethyl-daunomicinone 0.39%) were accomplished as in Example 1, obtaining 26.6 g of crystal 4-demethoxydaunomycinone. Overall yield 74.7%, HPLC purity 97.9%.(containing 7-deoxy-4-demethoxydaunomycinone 0.24% and 4-demethyl-daunomicinone 0.35%).
¹Starting material contains 0.25% of 4-demethyl-daunomicinone

### Example 4

A solution of 4-demethyldaunomycinone-4-triflate¹ (50.00 g, 96.7 mmol) and dichlorobis(triphenylphosphine)palladium(II) (1.36 g, 1.93 mmol) in dimethylformamide (1130 ml) was prepared at 15° C., under a nitrogen stream. A solution of triethylsilane (12.93 g, 111.2 mmol) and 2,6-dimethylpyridine (11.93 g, 111.0 mmol) in dimethylformamide (1080 ml) was added dropwise over 1.5 h. After 3.5 hours, the reaction was complete, and the work up of the mixture and purification of crude product (purity 94%, containing 7-deoxy-4-demethoxydaunomycinone 3.0%, Idarubicin aglycone bis-anhydro 0.1% and 4-demethyl-daunomicinone 0.44%) were accomplished as in Example 1, affording 4-demethoxydaunomycinone in 88.2% overall yield and in purity of 98% by HPLC. (containing 7-deoxy-4-demethoxydaunomycinone 1.0% and 4-demethyl-daunomicinone 0.28%)
¹Starting material contains 0.25% of 4-demethyl-daunomicinone

### Example 5

A solution of 4-demethyldaunomycinone-4-triflate¹ (1.88 g, 3.64 mmol) and dichlorobis(triphenylphosphine)palladium(II) (0.0513 g, 0.0731 mmol) in dimethylformamide (44 ml) was prepared at 46° C., under a nitrogen stream. A solution of triethylsilane (0.4645 g, 4.00 mmol) and triethylamine (0.4074 g, 4.02 mmol) in dimethylformamide (34 ml) was added dropwise over 1 h. When the conversion of 4-demethyldaunomycinone-4-triflate arrested, a solution of triethylsilane (0.0524 g, 0.45 mmol) and triethylamine (0.0423 g, 0.42 mmol) in dimethylformamide (10 ml) was added. After 0.5 h, the reaction mixture was cooled to 28° C., treated with glacial acetic acid (1.61 ml), heated to 50° C., and treated with hydrochloric acid 37% (0.75 ml). After 1.5 h, H₂O (140 ml) was added dropwise (1.5 h) at 50° C. and the mixture was slowly cooled to room temperature. The crude product was recovered by filtration, washed with a mixture of H₂O (14 ml) and methanol (14 ml) and dried at 50° C. under vacuum, affording crude 1.05 g (73% yield) of 4-demethoxydaunomycinone HPLC purity 93.3%, containing 7-deoxy-4-demethoxydaunomycinone less than 0.1%, 4-demethyldaunomycinone 0.2%, Idarubicin aglycone bis-anhydro 3.6%).
¹Starting material contains less than 0.10% of 4-demethyl-daunomicinone.

### Example 6

A solution of 4-demethyldaunomycinone-4-triflate¹ (50.00 g, 96.7 mmol) and dichlorobis(triphenylphosphine)palladium(II) (1.36 g, 1.93 mmol) in dimethylformamide (1100 ml) was prepared at 46° C., under a nitrogen stream. A solution of triethylsilane (13.50 g, 116.1 mmol) and 2,6-dimethylpyridine (11.41 g, 106.5 mmol) in dimethylformamide (1025 ml) was added dropwise over 1 h. When the conversion of 4-demethyldaunomycinone-4-triflate arrested (40 min.), a solution of triethylsilane (0.57 g, 4.92 mmol) in dimethylformamide (50 ml) was added. After 20 minutes, the reaction was complete and the work up of the mixture was accomplished as in Example 5, affording crude 4-demethoxydaunomycinone (32.6 g, HPLC purity 95.0%, containing 7-deoxy-4-demethoxydaunomycinone 0.47% Idarubicin aglycone bis-anhydro 0.3%, 4-demethyldaunomycinone-4-triflate 0.4% and 4-demethyl-daunomicinone less than 0.1%, 87% yield).
¹Starting material contains less than 0.10% of 4-demethyl-daunomicinone.

The product was then crystallized using a 4:6 mixture of toluene and THF (11) to dissolve the product at reflux followed by cooling for 2 hours to 25° C. and maintaining the mixture overnight at this temperature. After filtration, washing and drying, the pure product was obtained. HPLC purity 98.7% (containing 7-deoxy-4-demethoxydaunomycinone 0.11%, and 4-demethyl-daunomicinone less than 0.10%).

### Example 7

To a solution of 4-demethoxy-daunomycinone (1 g) in 230 ml of anhydrous chloroform, (2.2 g) of 2,3,6-trideoxy-3-trifluoroacetamido-4-O-trifluoroacetyl-α-L-lyxopyranosyl-chloride, (2 g) of HgO, (0.5 g) of HgBr₂, and (15 g) of 5 A molecular sieve were added under stirring. The suspension was stirred in the dark for 24 hours, filtered, concentrated in vacuo and the residue dissolved in 350 ml of methanol. The solution was left overnight at room temperature. After evaporation of the solvent the residue was chromatographed on (20 g) silica gel, eluting first with chloroform:acetone 19:1 to give 0.55 g of a (−)-daunosaminyl-4-demethoxydaunomycinone-N-trifluoroacetate, which was then dissolved in 40 ml of 0.1N NaOH and kept at room temperature for 30 minutes. The solution was brought to pH 8 with HCl, and extracted with chloroform. Evaporation of the left a residue that was taken up in a little chloroform-methanol. Methanolic 0.1N HCl was added to adjust the pH to 4.5, after which sufficient ethylether was added to precipitate α-(−)-daunosaminyl-4-demethoxydaunomycinone hydrochloride.

HPLC

Column: Hypersil BDS-C18 3μm − 100 × 4.6 mm
Mobil phase: Buffer (*): THF = 70:30
Flow: 1.5 ml/mm
Temperature: 50° C.
Wavelength: 254 nm
Run: 30 minutes
(*) Buffer: 2.9 g sodium laurylsulfate - 1000 ml of water - 2.3 g
phosphoric acid 85%

| | Retention | |
|---|---|---|
| Product | time | RRT |
| 4-demethyl-daunomycinone-4-Triflate | 9.10 | 2.4 |
| 4-demethoxy-daunomycinone | 3.75 | 1.0 |
| 4-demethyl-daunomycinone | 4.44 | 1.2 |
| 7-deoxy-4-demethoxy-daunomycinone | 6.09 | 1.6 |
| Idarubicin aglycone bis-anhydro | 13.77 | 3.7 |

What is claimed is:

1. A process for the preparation of idarubicin aglycone (4-demethoxydaunomycinone) of formula I 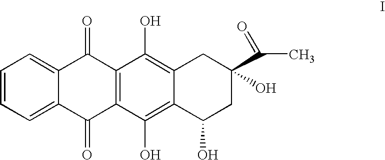
comprising the steps of
(a) combining the corresponding sulfonate of formula II, 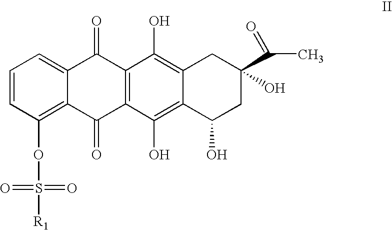
with a first polar aprotic organic solvent and with a metal catalyst or co-catalyst to obtain a first solution;
(b) combining about 0.9 to about 1.3 mole equivalents of the silyl reagent of the formula R₂R₃R₄SiH with a base, and a second polar aprotic organic solvent, with or without a protic solvent to obtain a second solution;
(c) adding the second solution of step (b) to the first solution of step (a) to obtain a mixture, and
(d) adding a solution of the silyl reagent of the formula R₂R₃R₄SiH in a second polar aprotic organic solvent to the mixture of step (c), when no more than 96% area by HPLC of the sulfonate of formula II has reacted, and
(e) maintaining the mixture obtained in step (d) for at least 20 minutes;
(f) quenching the mixture maintained in step (e), and
(g) recovering the idarubicin aglycone of the formula I; wherein R₁ is C₁₋₁₀ alkyl, C₁₋₁₀ alkyl substituted with halogen, an aryl group, an aryl group substituted with halogen or an electron withdrawing group, and R₂, R₃, and R₄ are independently branched or linear C₁₋₄ alkyl, aryl, heteroaryl groups or polymethylsiloxane.

2. The process of claim 1, wherein R₁ is C₁₋₁₀ alkyl fully substituted with halogen.

3. The process of claim 2, wherein R₁ is CF₃.

4. The process of claim 1, wherein R₂, R₃ and R₄ are the same alkyl groups.

5. The process of claim 4, wherein R₂, R₃ and R₄ are ethyl groups.

6. The process of claim 1, wherein the amount of silyl reagent of the formula R₂R₃R₄SiH in step (b) is about 1.15 mole equivalent per mole equivalent of the sulfonate of formula II.

7. The process of claim 1, wherein the second solution of step (b) is added to the first solution of step (a) in a dropwise manner.

8. The process of claim 7, wherein the dropwise addition is done over a period of about 20 minutes to about 1.5 hours.

9. The process of claim 1, wherein the second solution of step (b) also contains a protic solvent.

10. The process of claim 9, wherein the protic solvent is either C₁₋₅ alcohol or water.

11. The process of claim 10, wherein the C₁₋₅ alcohol is methanol.

12. The process of claim 9, wherein the protic solvent is water.

13. The process of claim 9, wherein the amount of the protic solvent is about 0.1 mole equivalents to about 5 mole equivalents per mole equivalent of the sulfonate of formula II.

14. The process of claim 1, wherein the temperature in step (a) is from about 10° C. to about 46° C.

15. The process of claim 1, wherein the first polar aprotic organic solvent used in step (a) is selected from the group consisting of amide, ether and ketone.

16. The process of claim 15, wherein the first polar aprotic organic solvent is selected from the group consisting of dimethylformamide (DMF), dimethylacetamide, N-methylpyrrolidinone (NMP), tetrahydrofuran (THF), 2-methyl-THF, N-methylpiperidone and acetone.

17. The process of claim 16, wherein the first polar aprotic organic solvent is DMF.

18. The process of claim 1, wherein a metal co-catalyst is used in step (a).

19. The process of claim 18, wherein the metal co-catalyst is dichlorobis(triphenylphosphine)Ni(II), dichlorobis(triphenylphosphine)Pd(II), or Pd(II)(OAc)₂ in the presence of triphenyl phosphine.

20. The process of claim 19, wherein the metal co-catalyst is dichlorobis(triphenylphosphine)Pd(II).

21. The process of claim 1, wherein the base in step (b) is a weak organic base.

22. The process of claim 21, wherein the base is a hindered weak organic base.

23. The process of claim 22, wherein the base is selected from the group consisting of pyridine, 2,6-dimethylpyridine, diisopropylethylamine, triethylamine, tributylamine, imidazole.

24. The process of claim 23, wherein the base is 2,6-dimethylpyridine.

25. The process of claim 1, wherein the second polar aprotic organic solvent in step (b) is the same as the first aprotic organic solvent in step (a).

26. The process of claim 1, wherein the mixture obtained in step (d) is maintained for about 20 minutes to about 2 hours, depending on the reaction temperature.

27. The process of claim 1, wherein the quenching reagent used in step (f) is an acidic aqueous solution.

28. The process of claim 27, wherein the acidic aqueous solution is selected from the group consisting of HCl, acetic acid, and ammonium chloride.

29. The process of claim 28, wherein the acidic aqueous solution is aqueous HCl.

30. The process of claim 1, further comprising crystallization of the product of step (g) from a mixture of a solvent and an anti-solvent.

31. The process of claim 30, wherein the solvent is a polar organic solvent.

32. The process of claim 31, wherein the polar organic solvent is selected from the group consisting of dichloromethane, acetone acetonitrile and THF.

33. The process of claim 30, wherein the anti-solvent is a non-polar organic solvent.

34. The process of claim 33, wherein the non-polar organic is either diisopropylether or toluene.

35. The process of claim 30, wherein the mixture of a solvent and an anti solvent contains acetonitrile with diisopropylether.

36. The process of claim 30, wherein the mixture of a solvent and an anti solvent contains THF with toluene.

37. The process of claim 30, wherein idarubicin aglycone contains less than about 1% area by HPLC, of undesired byproducts.

38. The process of claim 37, wherein idarubicin aglycone contains less than about 0.1% area by HPLC, of 4-hydroxy derivative of formula IV.

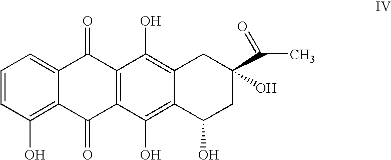

39. The process of claim 38, wherein idarubicin aglycone contains less than about 0.1% area by HPLC, of 4-hydroxy derivative of formula IV.

40. Idarubicin aglycone of formula I containing less than about 0.1% area by HPLC of 4-hydroxy derivative of formula IV.

41. The process of claim 1, further comprising converting the idarubicin aglycone to idarubicin hydrochloride.
